(19) United States
(12) Patent Application Publication
(10) Pub. No.: US 2004/0187610 A1
(43) Pub. Date: Sep. 30, 2004

(54) Sample solution measuring device

(75) Inventors: Koichiro Kawano, Kamakura-shi (JP); Yoshio Ishimori, Kawasaki-shi (JP); Yoshiaki Tsukumo, Tokyo (JP); Hajime Sudo, Matsudo-shi (JP); Yoshiyuki Isozaki, Tokyo (JP); Masahiro Kuwata, Kawasaki-shi (JP)

Correspondence Address: Finnegan, Henderson, Farabow,, Garrett & Dunner, L.L.P., 1300 I Street, N.W., Washington, DC, 20005-3315, US

(73) Assignee: KABUSHIKI KAISHA TOSHIBA

(21) Appl. No.: 10/812,933

(22) Filed: Mar. 31, 2004

(30) Foreign Application Priority Data
Mar. 31, 2003 (JP) P2003-95625

(51) Int. Cl. A61K 9/14; B01L 3/02

(52) U.S. Cl. 73/864.01

(57) ABSTRACT

A sample solution measuring device for detecting dissolved substances in a sample solution by measuring a potential difference between the sample solution and a reference solution, the sample solution measuring device, including: a sample solution container; a reference solution container; a substrate thin film is located between the sample solution container and the reference solution container and having a hole in which a bilayer lipid membrane is formed, where the sample solution and the reference solution come into contact with each other; a mechanism which forms the bilayer lipid membrane by ejecting a lipid solution; and, a mechanism which ejects a cleaning fluid to the substrate thin film.

### CROSS-REFERENCE TO RELATED APPLICATIONS

This application is based upon and claims the benefit of priority from prior Japanese Patent Application P2003-95625 filed on Mar. 31, 2003; the entire contents of which are incorporated by reference herein.

### BACKGROUND OF THE INVENTION

1. Field of the Invention

The present invention relates to a sample solution measuring device for measuring toxic substances existing in a sample.

2. Description of the Related Art

In the environment surrounding us today, there exist a number of substances hazardous to living organisms (toxic substances in a broad sense). For example, pollution of running water and underground water by so called toxic substances, such as cyanogens and cadmium besides trihalomethane in running water, dioxin in the atmosphere and the like, has occasionally been in all the headlines. In order to detect such toxic substances in real time, there has been proposed an “eco-sensor” which uses a bilayer lipid membrane as shown in FIG. 1 (see Japanese Patent Laid-Open No. 2001-91494). The eco-sensor includes a plurality of water tanks 117. In a part of a substrate thin film 103 that is a partition wall between the water tanks, provided is a hole 104 is formed, where a bilayer lipid membrane, on which toxic substances can act. The eco-sensor has a mechanism capable of sensing the toxic substances by obtaining a potential difference between a reference solution and a sample solution, both of which sandwich the substrate thin film 103 therebetween, by use of reference electrodes 133. Moreover, the bilayer lipid membrane is deteriorated with time and sensitivity thereof is weakened. Thus, in the eco-sensor, the membrane needs to be reproduced immediately before measurement. In order to realize the reproduction of the membrane, a lipid solution stored in a storage tank 131 is ejected into the hole 104 by an ejection part 130 using an inkjet mechanism and the bilayer lipid membrane is automatically formed.

However, lipid not used in formation of the bilayer lipid membrane adhere to the vicinity of the hole 104 and the water tanks 117. Thus, the lipid inhibits the formation of the bilayer lipid membrane. As shown in FIG. 2, a lipid solution not used in formation of the membrane in the hole 104 among lipid solution 105 ejected from a lipid solution ejection nozzle 102 remains adhering to the substrate thin film 103 as a contamination 106 by the lipid solution.

### SUMMARY OF THE INVENTION

A first aspect of the present invention is to provide a sample solution measuring device for detecting dissolved substances in a sample solution by measuring a potential difference between the sample solution and a reference solution, the sample solution measuring device, comprising: a) a sample solution container; b) a reference solution container; c) a substrate thin film is located between the sample solution container and the reference solution container and having a hole in which a bilayer lipid membrane is formed, where the sample solution and the reference solution come into contact with each other; d) a mechanism which forms the bilayer lipid membrane by ejecting a lipid solution; and, e) a mechanism which ejects a cleaning fluid to the substrate thin film.

A second aspect of the present invention is to provide a sample solution measuring device for detecting dissolved substances in a sample solution by measuring a potential difference between the sample solution and a reference solution, the sample solution measuring device, comprising: a) a sample solution container; b) a reference solution container; c) a substrate thin film is located between the sample solution container and the reference solution container and having a hole in which a bilayer lipid membrane is formed, where the sample solution and the reference solution come into contact with each other; d) a mechanism which forms the bilayer lipid membrane by ejecting a lipid solution; and, e) a mechanism which heats the hole to a melting temperature of the lipid or more, the mechanism being provided on the substrate thin film.

A third aspect of the present invention is to provide a sample solution measuring device for detecting dissolved substances in a sample solution by measuring a potential difference between the sample solution and a reference solution, the sample solution measuring device, comprising: a) a sample solution container; b) a reference solution container; c) a substrate thin film is located between the sample solution container and the reference solution container and having a hole in which a bilayer lipid membrane is formed, where the sample solution and the reference solution come into contact with each other; d) a mechanism which forms the bilayer lipid membrane by ejecting a lipid solution; and, e) a mechanism which vibrates the hole being provided on the substrate thin film.

A forth aspect of the present invention is to provide a sample solution measuring device for detecting dissolved substances in a sample solution by measuring a potential difference between the sample solution and a reference solution, the sample solution measuring device, comprising: a) a sample solution container; b) a reference solution container; c) a substrate thin film is located between the sample solution container and the reference solution container and having a hole in which a bilayer lipid membrane is formed, where the sample solution and the reference solution come into contact with each other; d) a mechanism which forms the bilayer lipid membrane by ejecting a lipid solution; and, e) a mechanism which heats the sample solution and the reference solution to a melting temperature of the lipid or more.

A fifth aspect of the present invention is to provide a sample solution measuring device for detecting dissolved substances in a sample solution by measuring a potential difference between the sample solution and a reference solution, the sample solution measuring device, comprising: a) a sample solution container; b) a reference solution container; c) a substrate thin film is located between the sample solution container and the reference solution container and having a hole in which a bilayer lipid membrane is formed, where the sample solution and the reference solution come into contact with each other; d) a mechanism which forms the bilayer lipid membrane by ejecting a lipid solution; and, e) a mechanism which stirs any of the sample solution and the reference solution.

### BRIEF DESCRIPTION OF THE DRAWINGS

FIG. 1 is a schematic diagram of a conventional sample solution measuring device.

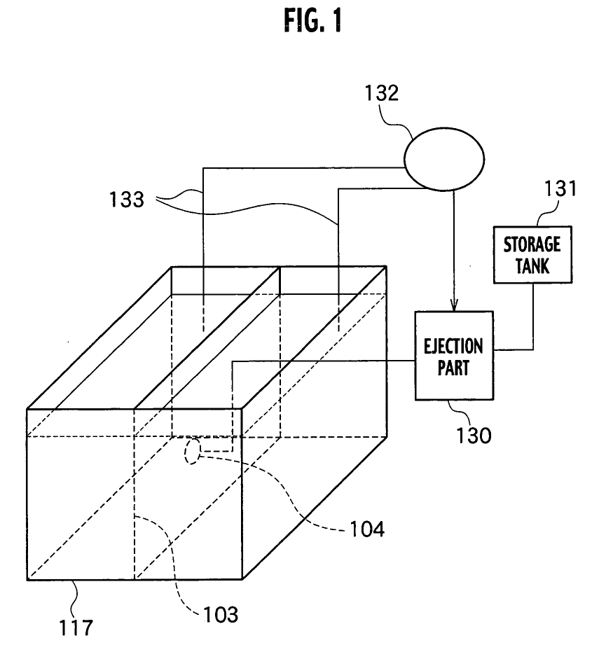

FIG. 2 is an enlarged view of a periphery of a hole shown in FIG. 1.

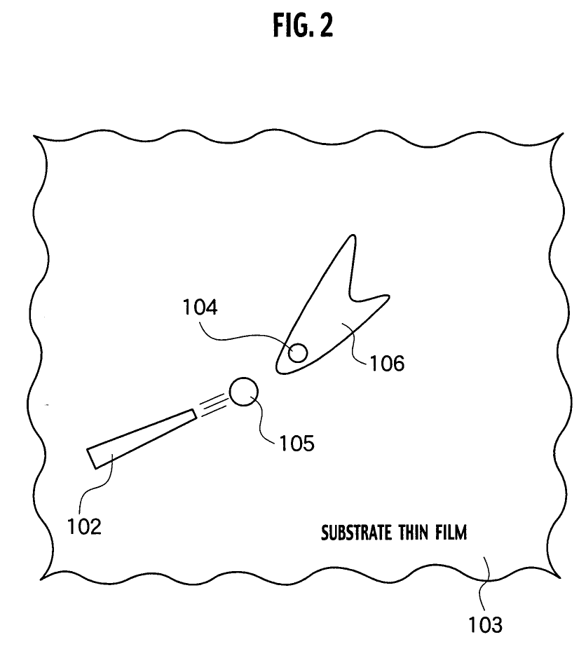

FIG. 3 is a schematic diagram of a sample solution measuring device according to a first embodiment.

FIG. 4 is a schematic diagram of a cleaning nozzle of the sample solution measuring device according to the first embodiment.

FIG. 5 is a view in the case of lowering a water level of the sample solution measuring device according to the first embodiment.

FIG. 6 is an enlarged view of a substrate thin film of a sample solution measuring device according to a second embodiment (No. 1).

FIG. 7 is an enlarged view of a substrate thin film of the sample solution measuring device according to the second embodiment (No. 2).

FIG. 8 is a schematic diagram of a sample solution measuring device according to a third embodiment (No. 1).

FIG. 9 is a schematic diagram of a sample solution measuring device according to the third embodiment (No. 2).

FIG. 10 is a schematic diagram of a sample solution measuring device according to a fourth embodiment.

### DETAILED DESCRIPTION OF THE INVENTION

Various embodiments of the present invention will be described with reference to the accompanying drawings. It is to be noted that the same or similar reference numerals are applied to the same or similar parts and elements throughout the drawings, and the description of the same or similar parts and elements will be omitted or simplified.

(First Embodiment)

In a first embodiment, description will be given of a sample solution measuring device which removes an excess lipid that adhere to a periphery of a hole of a substrate thin film. In the first embodiment, a cleaning fluid is ejected to the hole and unnecessary lipid is removed.

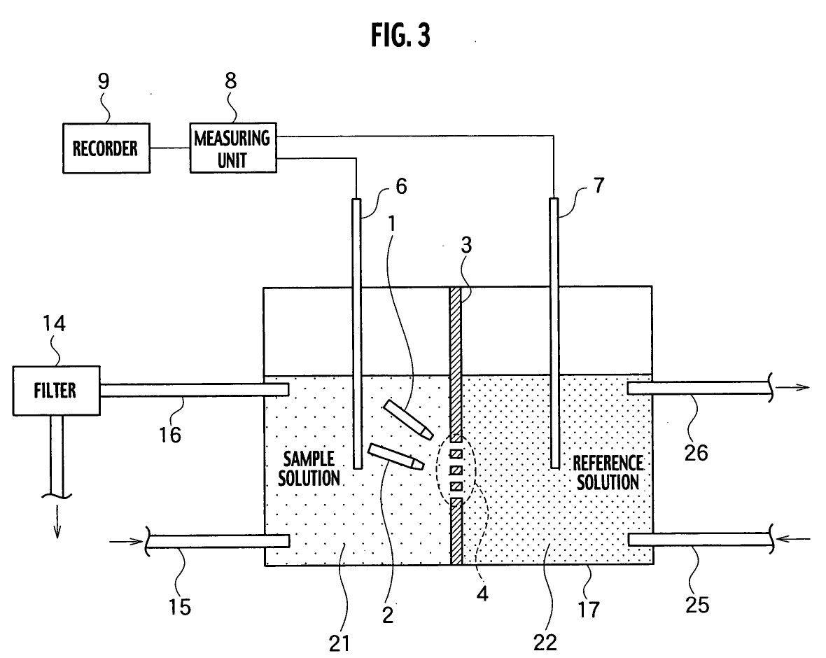

As shown in FIG. 3, the sample solution measuring device according to the first embodiment includes a sample solution container 21, a reference solution container 22, a substrate thin film 3 which is located between the sample solution container 21 and the reference solution container 22 and has a hole 4 in which a bilayer lipid membrane is formed, a lipid solution ejection nozzle 2, a cleaning nozzle 1. Specifically, on the bilayer lipid membrane, a sample solution and a reference solution come into contact with each other.

An electrode 6 is provided in the sample solution container 21 and this electrode 6 is inserted into the sample solution. Similarly, an electrode 7 is provided in the reference solution container 22 and this electrode 7 is inserted into the reference solution. By use of these electrodes 6 and 7, a potential difference (that is, a membrane potential) between the sample solution and the reference solution, which are separated from each other by the bilayer lipid membrane, can be measured by a measuring unit 8 such as an electrometer, for example. Data obtained by this measuring unit 8 can be recorded by a recorder 9.

The sample solution container 21 is configured to be capable of injecting and draining the sample solution. The injection and drainage of the sample solution are performed, for example, through at least one distribution port provided in a wall of the sample solution container 21. The sample solution is injected into the sample solution container 21 through the distribution port and the sample solution is measured. Thereafter, the measured sample solution can be drained from the distribution port described above or a different distribution port. In the sample solution measuring device according to the first embodiment, it is preferable that the injection and drainage of the sample solution to and from the sample solution container 21 are simultaneously and continuously performed by providing a plurality of distribution ports, that is inlets and outlets of the sample solution. As shown in FIG. 3, a sample solution inlet 15 is provided in a lower portion of the sample solution container 21 and a sample solution outlet 16 is provided in an upper portion thereof Accordingly, flows of the sample solution are generated in a periphery of the bilayer lipid membrane. Thus, the sample solution continuously comes into contact with the bilayer lipid membrane. In such a manner, the sample solution, as flows, is allowed to continuously come into contact with the bilayer lipid membrane and the potential difference between the sample solution and the reference solution is measured. Thus, dissolved matter in the sample solution can be continuously detected.

Meanwhile, the reference solution container 22 can be also similarly configured to be capable of injecting and draining the reference solution. Preferably, as shown in FIG. 3, a reference solution inlet 25 is provided in a lower portion of the reference solution container 22 and a reference solution outlet 26 is provided in an upper portion thereof. Thus, flows of the reference solution can be generated in the reference solution container 22.

The substrate thin film 3 is located between the sample solution container 21 and the reference solution container 22 and is formed of, for example, poly-tetra-fluoro-ethylene ((-CF2-CF2-)n) with a thickness of about 25 μm. The substrate thin film 3 has one hole 4 or more and the bilayer lipid membrane is formed in this hole 4.

The lipid solution ejection nozzle 2 is an inkjet-type device, which ejects a lipid solution to form the bilayer lipid membrane in the hole 4. In this event, the substrate thin film 3, in which the bilayer lipid membrane is formed, becomes a target on which lipid droplets are ejected by the inkjet mechanism. Thus, the substrate thin film 3 is contaminated by lipid. It is verified by experiments that the contamination of the substrate thin film 3 by the lipid makes it difficult for the bilayer lipid membrane to be formed. Thus, it is required to clean the substrate thin film 3 immediately before the bilayer lipid membrane is formed.

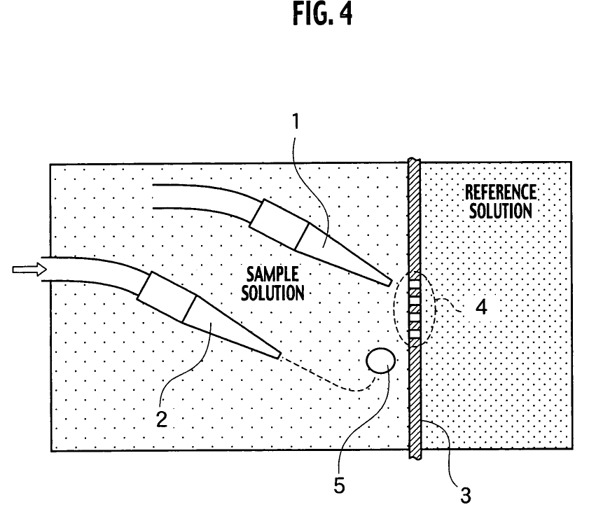

The cleaning nozzle 1 shown in FIGS. 3 and 4 ejects the cleaning fluid to the substrate thin film 3. And the cleaning nozzle washes off the contamination by the lipid, which adheres to the substrate thin film 3, by use of a jet stream of a cleaning fluid. It is preferable that cleaning of the lipid is performed every fixed period of time or immediately before measurement of the sample solution. Moreover, as the cleaning fluid to be used, any fluids can be used as long as the fluids can wash the bilayer lipid membrane. As preferable cleaning fluids, water, hot water and the like are enumerated. In some cases, a solvent of the sample solution, the sample solution, the reference solution or the like can be also used as the cleaning fluid.

Moreover, in general, lipid used in an eco-sensor are dissolved at 20 to 30° C. If a temperature of the jet stream upon arrival thereof at the substrate thin film 3 is equal to this melting temperature or more, dissolution of the lipid further increases an effect of cleaning. In order to set the cleaning fluid at the melting temperature of the lipid, for example, a heating unit such as a heating wire can be provided in the cleaning nozzle 1.

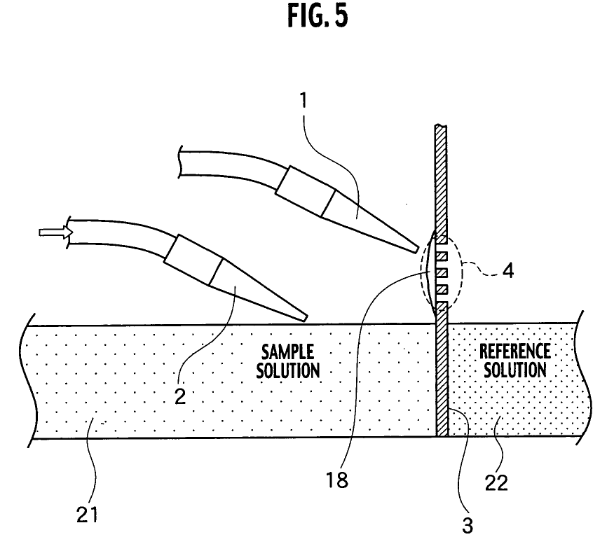

Moreover, as to the jet stream flowing in the cleaning, the stronger the force thereof is, the more the effect of cleaning the adhering lipid is increased. In FIG. 5, a sample solution level in a water tank is lowered in cleaning. As shown in FIG. 5, by cleaning the hole 4 in midair, the cleaning of the lipid can be performed while maintaining the force of the jet stream from the cleaning nozzle 1 without losing its kinetic energy to the sample solution in the water tank.

It is preferable that the cleaning nozzle 1 is placed in an ascending flow in the sample solution container 21 or the reference solution container 22 and cleaning of contamination by lipid is performed in the ascending flow. Usually, a specific gravity of a solution for forming the bilayer lipid membrane is smaller than that of the sample solution. This is because, in the case described above, lipid of the washed bilayer lipid membrane, an excess film forming solution arising in film formation and a contaminated cleaning fluid are encouraged to come up to the surface of the solution in the container. Thus, it becomes easy to drain the elements described above from the system.

The ascending flow can be generated by any method suited to accomplishing an object. For example, as shown in FIG. 3, the sample solution inlet 15 and the sample solution outlet 16 are provided in the lower and upper portions of the sample solution container 21, respectively. Thus, the ascending flow can be generated by allowing the sample solution in the periphery of the hole 4, to which the contamination adheres, to ascend. Moreover, the ascending flow can be also generated in the periphery of the hole 4, to which the contamination adheres, by providing a passage in the sample solution container 21 by use of a partition plate and the like. Thus, cleanliness of the substrate thin film 3 is secured.

In FIG. 3, the excess bilayer lipid membrane forming solution, the contaminated cleaning fluid and the like, all of which come up to the surface of the sample solution, are drained from the sample solution outlet 16. The drained solution can be recovered by separation means such as a filter 14, for example, according to need.

Note that the bilayer lipid membrane 18 according to the first embodiment is used to detect the presence of the dissolved substance and its concentration in the sample solution by using some kind of changes of the bilayer lipid membrane (for example, changes in a membrane potential, an electric capacity, ion permeability, light emission, heat generation, absorption of heat and the like) which are caused by an action of a dissolved substance in the sample solution. As such a bilayer lipid membrane, enumerated are: a bilayer lipid membrane substantially including only lipid; a bilayer lipid membrane having molecules of various proteins, sugar and the like, which adhere thereto and are mixed therein; and the like. The kinds and amounts of the lipid, proteins and sugar, a method for preparing the bilayer lipid membrane and the like are appropriately selected. Thus, it is possible to manufacture various sensors corresponding to specific contents including a measurement purpose, a sample solution and the like. As a preferable bilayer lipid membrane in the sample solution measuring device according to the first embodiment, enumerated are: a bilayer lipid membrane in which proteins, such as antibodies, or sugar adheres to or is mixed with quasi-lipid, such as mono-olein and tri-olein, or phospholipid; and the like.

By use of the sample solution measuring device according to the first embodiment, the unnecessary lipid, which adhere to the hole 4 of the substrate thin film 3, can be removed by using the cleaning nozzle 1. Thus, stability and reproducibility of the bilayer lipid membrane can be secured. In addition, it is possible to perform a wide range of detection and quantitative determination of toxic substances without maintenance and in real time.

Note that the lipid solution ejection nozzle 2 which ejects the lipid solution by use of the jet stream and the like is included in a mechanism of forming a bilayer lipid membrane by ejecting a lipid solution. The cleaning nozzle 1 which ejects the cleaning fluid by use of the jet stream and the like is included in a mechanism of ejecting a cleaning fluid to an unnecessary lipid, which adhere to a hole of a substrate thin film.

(Second Embodiment)

In a second embodiment, similarly to the first embodiment, description will be given of a sample solution measuring device which removes an excess lipid that adheres to a periphery of a hole of a substrate thin film. In the second embodiment, the substrate thin film is heated or vibrated to remove unnecessary lipid.

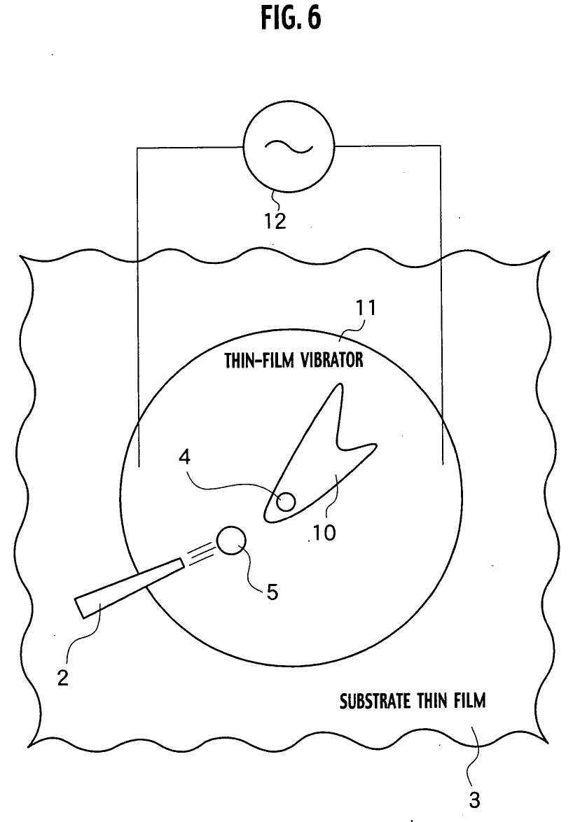

As shown in FIG. 6, a substrate thin film 3 of the sample solution measuring device according to the second embodiment includes a thin-film heater 11 in a periphery of a hole 4. The thin-film heater 11 is composed of metal or ceramics. Besides the component described above, a substrate thin film 3, a lipid solution ejection nozzle 2 and the like are the same as those of the first embodiment. Thus, description thereof will be omitted herein. The thin-film heater 11 is energized by a power source 12 to heat the substrate thin film 3 and dissolve a contamination 10 by lipid adhering to the substrate thin film 3. Thus, unnecessary lipid in the periphery of the hole 4 can be removed. As described in the first embodiment, an excess bilayer lipid membrane forming solution, a contaminated cleaning fluid and the like, all of which come up to the surface of the sample solution, are drained from a sample solution outlet. The drained solution can be recovered by separation means such as a filter 14, for example, according to need.

The sample solution measuring device according to the second embodiment, which is shown in FIG. 6, may include the cleaning nozzle 1 described in the first embodiment, in addition to the thin-film heater 11, and remove the unnecessary lipid by utilizing both of the melting temperature and the jet stream.

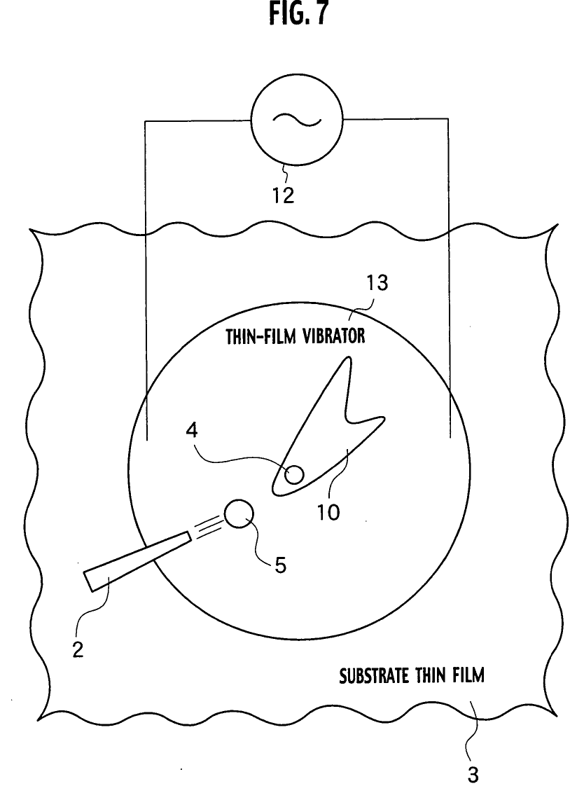

Moreover, as shown in FIG. 7, the substrate thin film 3 of the sample solution measuring device according to the second embodiment may include a thin-film vibrator 13 in the periphery of the hole 4. The thin-film vibrator 13 may be obtained by attaching an vibrator such as a piezoelectric element to the substrate thin-film 3. Alternatively, a hole may be formed in a thin vibrator itself. Besides the above, ultrasonic waves may be used. The thin-film vibrator 13 can liberate the contamination 10 by the lipid, which adheres to the substrate thin film 3, by vibrating the substrate thin film 3.

The sample solution measuring device according to the second embodiment, which is shown in FIG. 7, may include the cleaning nozzle 1 described in the first embodiment, in addition to the thin-film vibrator 13 and remove the unnecessary lipid by utilizing both of the vibration and the jet stream.

Furthermore, it is needless to say that the substrate thin film 3 of the sample solution measuring device according to the second embodiment may include both of the thin-film heater 11 and the thin-film vibrator 13.

By use of the sample solution measuring device according to the second embodiment, the unnecessary lipid, which adhere to the periphery of the hole 4, can be removed by using the thin-film heater 11 or the thin-film vibrator 13. Thus, stability and reproducibility of the bilayer lipid membrane can be secured. In addition, it is possible to perform a wide range of detection and quantitative determination of toxic substances without maintenance and in real time.

Note that the thin-film heater 11 attached to the substrate thin film is included in a mechanism of heating the sample solution and the reference solution to the melting temperature of lipid or more. The thin-film vibrator 13 attached to the substrate thin film is included in a mechanism of heating the hole to the melting temperature of lipid or more, which is provided on the substrate thin film.

(Third Embodiment)

In a third embodiment, description will be given of a sample solution measuring device which removes an excess lipid adhering to an entire water tank, without being limited to a periphery of a hole in a substrate thin film. In the third embodiment, unnecessary lipid is removed by heating a sample solution or a reference solution to a melting temperature of lipid or more.

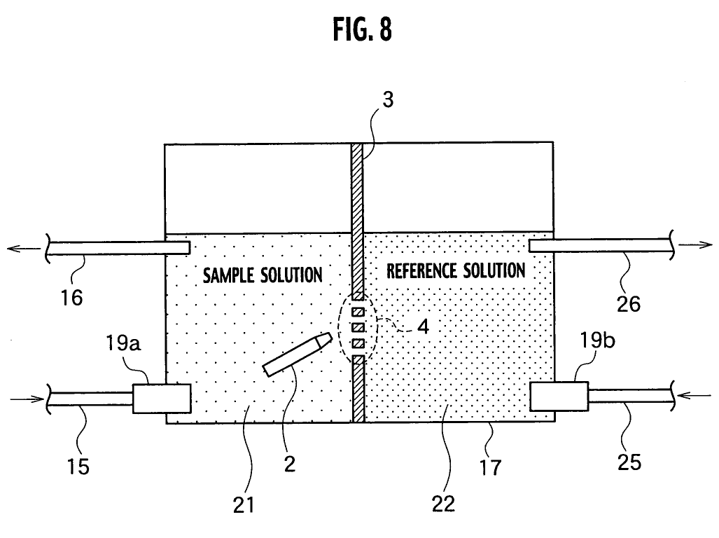

As shown in FIG. 8, the sample solution measuring device according to the third embodiment includes a heater 19a in the sample solution inlet 15. Thus, the sample solution introduced upon cleaning is heated by the heater 19a and introduced to a water tank 17. Moreover, the sample solution measuring device according to the third embodiment includes a heater 19b in the reference solution inlet 25. Thus, the reference solution introduced upon cleaning is heated by the heater 19b and introduced to the water tank 17. Besides the components described above, a lipid solution ejection nozzle 2, a substrate thin film 3, a sample solution outlet 16, a sample solution container 21, a reference solution container 22 and the like are the same as those of the first embodiment. Thus, description thereof will be omitted herein. The heater 19a heats the sample solution to the melting temperature of lipid or more, and the heater 19b heats the reference solution to the melting temperature of lipid or more. As described above, lipid is dissolved when heated to a certain temperature or more. Thus, a contamination by the lipid, which exists in the water tank 17, is dissolved and unnecessary lipid can be removed. As described in the first embodiment, an excess bilayer lipid membrane forming solution, a contaminated cleaning fluid and the like, all of which come up to the surface of the sample solution, are drained from the sample solution outlet 16. The drained solution can be recovered by separation means such as a filter, for example, according to need. Similarly, the solution drained from the reference solution outlet 26 can be also recovered by the separation means such as a filter. Note that, in FIG. 8, the heaters 19a and 19b are provided in both of the sample solution inlet 15 and the reference solution inlet 25. However, the sample solution measuring device according to the third embodiment may include the heater only in one of the inlets described above.

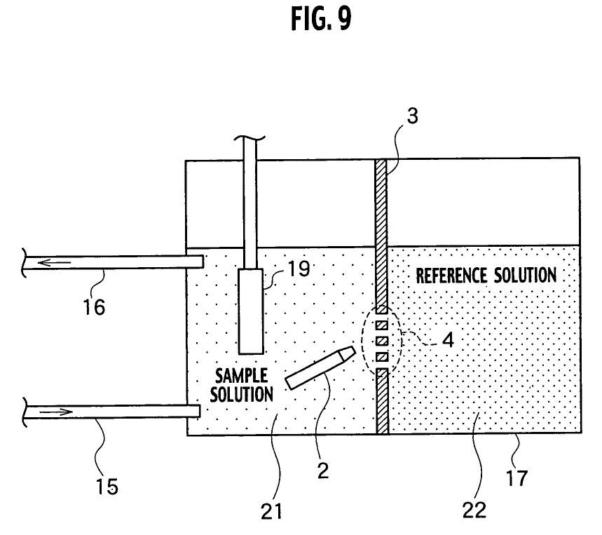

Moreover, as shown in FIG. 9, a heater 19 may be provided in the sample solution container 21 to heat the sample solution and the reference solution.

The sample solution measuring device according to the third embodiment may include the cleaning nozzle 1 described in the first embodiment, in addition to the heaters 19, 19a and 19b, and remove the unnecessary lipid by utilizing both of the melting temperature and the jet stream. Furthermore, the substrate thin film 3 of the sample solution measuring device according to the third embodiment may include the thin-film heater 11 or the thin-film vibrator 13, which are described in the second embodiment, and utilize the heating or vibration of the entire water tank and the substrate thin film 3.

By use of the sample solution measuring device according to the third embodiment, the unnecessary lipid, which exist in the water tank 17, can be dissolved and removed by heating the sample solution and the reference solution with the heaters 19, 19a and 19b. Thus, stability and reproducibility of the bilayer lipid membrane can be secured. In addition, it is possible to perform a wide range of detection and quantitative determination of toxic substances without maintenance and in real time.

Note that the heater 19a provided in the sample solution inlet, the heater 19b provided in the reference solution inlet and the heater 19 provided in the sample solution container are included in a mechanism of heating the sample solution and the reference solution to the melting temperature of lipid or more.

(Fourth Embodiment)

In a fourth embodiment, similarly to the third embodiment, description will be given of a sample solution measuring device which removes an excess lipid adhering to an entire water tank, without being limited to a periphery of a hole in a substrate thin film. In the fourth embodiment, unnecessary lipid is removed by stirring a sample solution and the like with a stirrer.

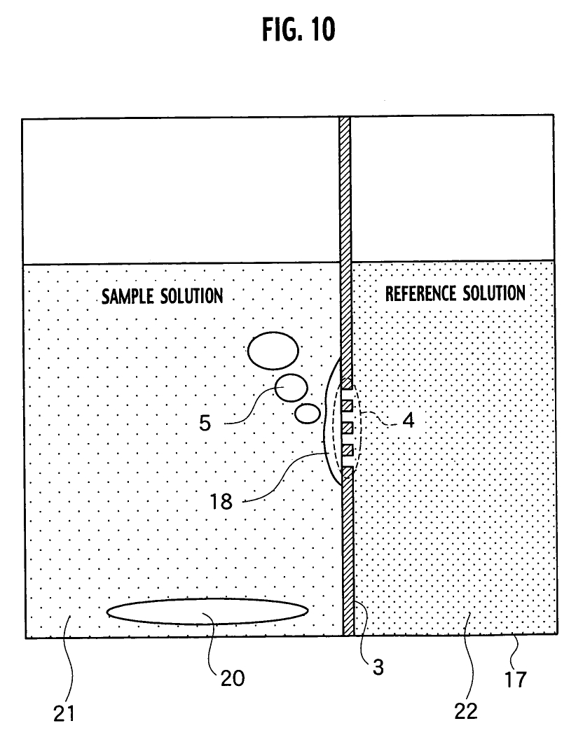

As shown in FIG. 10, the sample solution measuring device according to the fourth embodiment includes a stirrer 20 in the sample solution container 21. The other components thereof, such as a substrate thin film 3, are the same as those of the first embodiment. Thus, description thereof will be omitted herein. The stirrer 20 stirs the sample solution in the water tank 17 to cause convection and liberate the unnecessary lipid. In this event, an effect of washing away not only lipid on the substrate thin film 3 but also lipid in the entire water tank 17 can be expected. As described in the first embodiment, an excess bilayer lipid membrane forming solution, a contaminated cleaning fluid and the like, all of which come up to the surface of the sample solution, are drained from the sample solution outlet. The drained solution can be recovered by separation means such as a filter, for example, according to need. Although, the stirrer is provided in the sample solution container 21 in FIG. 10, it is needless to say that the stirrer may be provided in the reference solution container 22.

The sample solution measuring device according to the fourth embodiment may include the cleaning nozzle 1 described in the first embodiment, in addition to the stirrer 20, and remove the unnecessary lipid by utilizing both of the convection and the jet stream. Moreover, the substrate thin film 3 of the sample solution measuring device according to the fourth embodiment may include the thin-film heater 11 or the thin-film vibrator 13, which are described in the second embodiment, and utilize not only the convection but also the melting temperature or the vibration. Furthermore, the sample solution measuring device according to the fourth embodiment may include the heaters 19, 19a and 19b, which are described in the third embodiment, and utilize not only the convection but also the heating of the entire water tank 17.

By use of the sample solution measuring device according to the fourth embodiment, the unnecessary lipid, which exist in the water tank 17, can be removed by stirring the sample solution or the reference solution with the stirrer 20. Thus, stability and reproducibility of the bilayer lipid membrane can be secured. In addition, it is possible to perform a wide range of detection and quantitative determination of toxic substances without maintenance and in real time.

Note that the stirrer 20 provided in the sample solution container or the reference solution container is included in a mechanism of stirring the sample solution or the reference solution.

(Other Embodiments)

The present invention has been described according to the first to fourth embodiments described above. However, it should be understood that the present invention is not limited by the description and drawings, which constitute a part of this disclosure. Various alternative embodiments, embodiments and operational technologies will become apparent to those skilled in the art from this disclosure.

For example, in the first to fourth embodiments, the cleaning nozzle 1, the lipid solution ejection nozzle 2, the heater 19, the stirrer 20 and the like are described with reference to the drawings in which those elements are provided in the sample solution container 21. However, it is needless to say that the elements described above may be provided in the reference solution container 22.

Moreover, the sample solution measuring device according to the first to fourth embodiments may include the elements described in the respective embodiments in combination. For example, the cleaning nozzle 1, the thin-film vibrator 13 and the heaters 19, 19a and 19b are selected and the device may have a form including those selected elements. The respective elements described in the first to fourth embodiments can be appropriately selected in accordance with the kind of the sample solution and conditions of measurement.

Various modifications will become possible for those skilled in the art after receiving the teachings of the present disclosure without departing from the scope thereof.

What is claimed is:

1. A sample solution measuring device for detecting dissolved substances in a sample solution by measuring a potential difference between the sample solution and a reference solution, the sample solution measuring device, comprising:
a sample solution container;
a reference solution container;
a substrate thin film located between the sample solution container and the reference solution container and having a hole in which a bilayer lipid membrane is formed, where the sample solution and the reference solution come into contact with each other;
a mechanism which forms the bilayer lipid membrane by ejecting a lipid solution; and,
a mechanism which ejects a cleaning fluid to the substrate thin film.

2. The sample solution measuring device of claim 1, wherein a temperature of the cleaning fluid upon arrival at the substrate thin film is a melting temperature of the lipid or more.

3. The sample solution measuring device of claim 1, wherein water levels of the sample solution and the reference solution are positioned lower than the hole when the cleaning fluid is ejected to the substrate thin film.

4. The sample solution measuring device of claim 1, wherein the mechanism which ejects the cleaning fluid to the substrate thin film, is a cleaning nozzle which ejects a cleaning fluid by a jet stream.

5. A sample solution measuring device for detecting dissolved substances in a sample solution by measuring a potential difference between the sample solution and a reference solution, the sample solution measuring device, comprising:
a sample solution container;
a reference solution container;
a substrate thin film located between the sample solution container and the reference solution container and having a hole in which a bilayer lipid membrane is formed, where the sample solution and the reference solution come into contact with each other;
a mechanism which forms the bilayer lipid membrane by ejecting a lipid solution; and,
a mechanism which heats the hole to a melting temperature of the lipid or more, the mechanism being provided on the substrate thin film.

6. The sample solution measuring device of claim 5, wherein the mechanism which heats the hole to the melting temperature of the lipid or more is provided on the substrate thin film, is a thin-film heater attached to the substrate thin film.

7. A sample solution measuring device for detecting dissolved substances in a sample solution by measuring a potential difference between the sample solution and a reference solution, the sample solution measuring device, comprising:
a sample solution container;
a reference solution container;
a substrate thin film located between the sample solution container and the reference solution container and having a hole in which a bilayer lipid membrane is formed, where the sample solution and the reference solution come into contact with each other;
a mechanism which forms the bilayer lipid membrane by ejecting a lipid solution; and,
a mechanism which vibrates the hole being provided on the substrate thin film.

8. The sample solution measuring device of claim 7, wherein the mechanism which vibrates the hole is provided on the substrate thin film, is a thin-film vibrator attached to the substrate thin film.

9. A sample solution measuring device for detecting dissolved substances in a sample solution by measuring a potential difference between the sample solution and a reference solution, the sample solution measuring device, comprising:
a sample solution container;
a reference solution container;
a substrate thin film located between the sample solution container and the reference solution container and having a hole in which a bilayer lipid membrane is formed, where the sample solution and the reference solution come into contact with each other;
a mechanism which forms the bilayer lipid membrane by ejecting a lipid solution; and,
a mechanism which heats the sample solution and the reference solution to a melting temperature of the lipid or more.

10. The sample solution measuring device of claim 9, wherein the mechanism which heats the sample solution and the reference solution to the melting temperature of the lipid or more, is a thin-film heater attached to the substrate thin film.

11. The sample solution measuring device of claim 9, wherein the mechanism which heats the sample solution and the reference solution to the melting temperature of the lipid or more, is a heater provided in any of a sample solution inlet, a reference solution inlet, the sample solution container and the reference solution container.

12. A sample solution measuring device for detecting dissolved substances in a sample solution by measuring a potential difference between the sample solution and a reference solution, the sample solution measuring device, comprising:
a sample solution container;
a reference solution container;
a substrate thin film located between the sample solution container and the reference solution container and having a hole in which a bilayer lipid membrane is formed, where the sample solution and the reference solution come into contact with each other;
a mechanism which forms the bilayer lipid membrane by ejecting a lipid solution; and,
a mechanism which stirs any of the sample solution and the reference solution.

13. The sample solution measuring device of claim 12, wherein the mechanism which stirs any of the sample solution and the reference solution is a stirrer provided in any of the sample solution container and the reference solution container.
